(19) United States
(12) Patent Application Publication
(10) Pub. No.: US 2026/0344984 A1
(43) Pub. Date: Nov. 5, 2026

(54) Endoscope with rotary drive

(71) Applicant: Konstantin Bob, Weinheim (DE)

(72) Inventors: Konstantin Bob, Weinheim (DE); Andreas Gründl, Starnberg (DE)

(73) Assignee: Konstantin Bob, Weinheim (DE)

(21) Appl. No.: 19/119,971

(22) PCT Filed: Oct. 10, 2023
(86) PCT No.: PCT/EP2023/078082

§ 371 Date: Apr. 10, 2025

(30) Foreign Application Priority Data
Oct. 11, 2022 (DE) 10 2022 126 400.9

(51) Int. Cl. A61B 1/00; A61B 1/018; A61B 1/05; A61M 25/00; A61M 25/01

(52) U.S. Cl. CPC A61B 1/0016; A61B 1/018; A61B 1/05; A61M 25/0155; A61M 2025/0004; A61M 25/0147

(57) ABSTRACT

An endoscope has a substantially tubular shaft, and an endoscope head, which is arranged distally of the shaft and is able to be bent or folded relative to the shaft axis. A rotating mechanism is arranged between the endoscope head and the shaft and is configured to rotate the endoscope head relative to the shaft about the shaft axis. The rotating mechanism has the following: a base sleeve connected to the shaft in a torque-proof manner with respect to the shaft axis, a rotating sleeve connected to the endoscope head or to an intermediate portion in a torque-proof manner relative to the shaft axis, the rotating sleeve and the base sleeve being nested within one another, and a first cord having a distal cord portion which is attached to the rotating sleeve in such a way that the first cord can be wound or unwound around the rotating sleeve.

The present disclosure relates to an endoscope with a proximal, essentially tubular shaft, which is designed to be inserted into a patient's body and extends along a shaft axis, a substantially tubular steering portion, which is arranged distally of the shaft and is bendable with respect to the shaft axis, an endoscope head, which is arranged distally of the steering portion and comprises an optics and a working channel outlet, and a rotating mechanism configured to rotate the steering portion relative to the shaft about the shaft axis.

### BACKGROUND OF THE DISCLOSURE

Endoscopes are medical instruments for visual exploration of cavities in a patient's body. They basically have optical devices at the distal endoscope head, i.e. the end facing away from the user and respectively facing a patient's body, a proximal, i.e. user-facing, endoscope operating portion or endoscope handle and a flexible or rigid shaft that connects the endoscope head and the proximal endoscope operating portion or endoscope handle. Moreover, the endoscope usually has a working channel that extends from the proximal endoscope operating portion through the shaft to a working channel outlet formed at the endoscope head and allows extracorporeal introduction and use of a medical instrument such as, for example, forceps, scissors, a needle, a sling, a knife or the like. Many endoscopes further comprise deflectors, i.e., a steerable shaft portion located between the shaft and the endoscope head for enabling steering of the endoscope within a patient cavity.

Optionally, such endoscopes can be provided with additional capabilities, for example, by placing a cap or sleeve on the distal endoscope end/endoscope head on the radially outer side of the endoscope head, which is provided or equipped with specific functions/functional elements, whereby the endoscope can be used not only for exploration and/or as an access for therapeutic applications, but also as a minimally invasive instrument for executing a surgical procedure. Alternatively, it is also intended to equip special endoscopes for very specific medical applications integrally with such capabilities, such custom-made products, however, being only suitable for this particular application.

For example, various diagnostic and/or therapeutic procedures require imaging and/or, if required, therapeutic techniques on the patient's common bile duct, pancreatic duct, and hepatic ducts. Since the major duodenal papilla which is the common outlet of the bile and pancreatic ducts into the duodenum, protrudes laterally into the duodenum, conventional prograde (looking in the longitudinal direction of the endoscope) endoscopes are unsuitable for introducing and inserting surgical instruments into the bile duct, as there is not enough room to maneuver in the narrow duodenum (diameter 3 to 4 cm) to align their prograde optics and working channel in a sideways-looking position, since the typical bending radius of such devices is about 6 cm. In conventional prograde endoscopes, this bending radius or swiveling motion is often achieved by means of so-called “deflectors”, i.e. actively bendable shaft portions arranged between the shaft and the endoscope head, in particular directly in front of the endoscope head in the proximal direction.

### PRIOR ART

From the prior art (e.g. US 2010/228086 A) duodenoscopes specially designed for this purpose are known, which have lateral (sideways-looking) or retrograde (backward-looking) optics (also called “side optics”) as well as a sideways-facing or opening working channel. Alternatively, a so-called Albarran lever can be provided at a prograde (opening in the axial direction) outlet of the working channel of such duodenoscopes, which, when manually pivoted, enables targeted guidance/deflection of an instrument guided in the working channel. The lateral arrangement of the functional units, in particular optics and lighting on the endoscope head, enables imaging and treatment in the duodenum while making optimal use of the available space.

However, such endoscopes with side optics are very complex and expensive to manufacture and are therefore currently developed and manufactured as reusable devices. Moreover, the curved working channel of such endoscopes and the complex and undercut construction of the Albarran lever have proven difficult to sterilize in practice. This increases the risk of contamination of the endoscope and, as a result, the contamination of a surgical site or cavity of a patient in whom the endoscope is used. This can lead to serious inflammation at the surgical site or in the cavity, up to sepsis. Furthermore, such devices have the disadvantage that they can only be utilized for few very specific procedures in the duodenum, since neither the optics nor the working channel can be directed in the prograde direction. Navigation in a patient's body, especially in narrow patient cavities, is thus generally rather difficult with endoscopes that look sideways.

Furthermore, a folding mechanism for a fold-down or bendable holder of an endoscope head is known from DE 10 2018 110 620 A1, with a number of axially successive segments that can be controlled actively, i.e. manually, against each other in terms of angle. This design makes it possible to pivot the distal endoscope head in a very narrow radius into a radial alignment in such a way that the distal end face of the endoscope head does not or only slightly projects radially beyond the outer circumference of the endoscope shaft. However, the disadvantage of this mechanism is that it is only suitable for folding down in a single specific direction, thus limiting the range of applications of the associated endoscope. Furthermore, it can be problematic if the folding mechanism is misaligned at the site to be examined in a patient's cavity.

WO 201270781 A2 discloses a rotating mechanism that connects a flexible endoscope shaft to a bending mechanism arranged distally thereto in such a way that they can rotate about the shaft axis in relation to each other, with various drives being provided. However, these drives are very complex and require a lot of space and/or, as in the case of a drive via a cord, for example, are prone to failure and fragile.

### SUMMARY OF THE DISCLOSURE

The underlying task of the disclosure is to avoid or reduce disadvantages of the prior art. In particular, an endoscope with a steerable endoscope head and a simple, robust rotary drive is to be provided.

The task is solved by an endoscope according to claim 1. Advantageous embodiments are subject of the sub-claims.

More precisely, the task is solved by means of an endoscope comprising a substantially tubular shaft which is designed for insertion into a patient's body and extends along a shaft axis, an endoscope head, which is arranged distally from the shaft, is bendable or can be folded down with respect to the shaft axis, and comprises a camera as well as an outlet of a working channel and a rotating mechanism that is arranged between the endoscope head and the shaft and is configured to rotate the endoscope head about the shaft axis with respect to the shaft. The rotating mechanism has a base sleeve that is connected to the shaft in a torque-proof manner with regard to the shaft axis and a rotating sleeve that is coupled to the endoscope head in a torque-proof manner with regard to the shaft axis, or an intermediate portion, the rotating sleeve and the base sleeve being nested within one another. The rotating mechanism further comprises a first cord having a distal cord portion that is attached to the rotating sleeve such that the first cord can be wound or unwound around the rotating sleeve, the base sleeve forming in its side wall or on/in a surface of the side wall that is radially opposite (i.e. opposed) to the rotating sleeve, a first guide portion in the form of a groove- or slot-shaped reception, in which the first cord is guided. In particular, the groove- or slot-shaped reception is closed at least on one side facing the rotating sleeve, e.g. by an integral wall portion of the base sleeve and/or by a wall portion that is formed separately from the base sleeve but is fixedly connected thereto. Such a separate wall portion that, however, is firmly connected to the base sleeve may be, for example, a wall portion, such as a small tube, in which the first cord is guided, that is firmly inserted into the groove- or slot-shaped reception of the base sleeve (e.g. by soldering, gluing or the like). At a distal end of the first guide portion, the base sleeve forms a first distal passage through which the first cord is guided from the first guide portion to the rotating sleeve. That is, the passage connects the groove- or slot-shaped reception and possibly an element inserted therein, such as the small tube, to a side of the base element facing the rotating sleeve, so that the cord is guided from the groove- or slot-shaped reception through the passage to the rotating sleeve. In particular, the passage may be a hole in the integral or separate wall portion, but firmly attached to the base sleeve. Alternatively, the passage may be formed through a gap between a distal end of the groove- or slot-shaped reception and a distal end of the separate wall portion (e.g., the distal end of the tube) inserted therein. Therein, the first cord may be wound around the rotating sleeve.

In other words, an endoscope is provided that has a shaft and a distal endoscope portion with a bendable or foldable endoscope head and a rotary drive between the shaft and the distal endoscope portion. The rotary drive is a cord drive, which is based on the fact that a first cord is provided, which is wound in the circumferential direction onto a rotating sleeve (also known as a spool), and unwinding of the first cord and thus rotation of the rotating sleeve is achieved by pulling on the first cord. For this purpose, the rotating sleeve is mounted rotatably and preferably axially fixed on a base sleeve which connects the rotating mechanism to the shaft.

Terms such as “axial”, “circumferential” and “radial” always refer to the shaft axis. The shaft can be rigid or flexible. The endoscope head may moreover have a lighting device and/or a rinsing device and/or other elements that are connected to associated lines. Nested inside one another means that the two sleeves (rotating sleeve and base sleeve) overlap axially, at least partially, or that one of the two sleeves is inserted into the other one of the two sleeves.

The rotating mechanism is operated by pulling on the first cord that is wound around the rotating sleeve. This causes the cord to unwind from the rotating sleeve, driving it in the circumferential direction, i.e. rotating it. The endoscope head is bendable or foldable, in particular with respect to the shaft axis, especially to exactly one side (i.e. in a single radial direction).

The rotating mechanism of the protruding endoscope has a particularly simple configuration and is thus inexpensive to manufacture. Furthermore, the rotating mechanism is low-friction and thus easy to operate. In addition, the first cord is guided and deflected in a particularly safe manner so that it is guided securely. This minimizes the risk of the cord tearing.

Preferably, a second cord is provided, which is guided in a distally extending direction through a second guide portion in the form of a groove- or slot-shaped reception in the base sleeve and is wound or can be wound around the rotating sleeve in a circumferential direction opposite to the first cord. Preferably, the base sleeve forms a second distal passage at the distal end of the second guide portion, which is spaced apart from the first passage in the circumferential direction and through which the second cord is guided.

In other words, the second guide portion and the second cord run in a mirror image to the first guide portion and the first cord. This provides a particularly simple and reliable means of driving the rotating sleeve in both directions of rotation. In other words, depending which of the two cords (the first or second cord) is pulled, the rotating sleeve is turned in different directions. In this design, at least one of the two cords is always wound around the rotating sleeve. In a neutral position, both cords are wound around the rotating sleeve by at least 180°.

The first and/or second cord is/are preferably designed and dimensioned so that it is/they are or can be wound around the rotating sleeve by at least 360°. This allows the endoscope head to be rotated completely. The rotating sleeve can have one or more mounting holes, in particular radial holes, in which the first and/or second cord is fixed. This allows for a simple and secure attachment of the cords to the rotating sleeve.

Preferably, the first and/or the second cord each is/are guided in a capillary tube attached in or on the first or second guide portion of the base sleeve in a manner that is essentially fixed in position. This allows the first and/or second cord to be held particularly securely in the respective guide portion. Jumping out of the respective guide portion under tension and, as a result, jamming between the base sleeve and another component may thus be prevented. The capillary tube can have a wall thickness of 0.4 mm, for example, and can be attached, e.g. glued or soldered, in the first or second guide portion.

Preferably, the first and/or second guide portion is/are open on a radial outer side of the base sleeve, in particular along its entire length. This makes it particularly easy to assemble the first and respectively second cord and, if necessary, the capillary tubes in the respective guide portion.

Preferably, at least a portion of the first and/or second guide portion extends directly proximal to the first and/or second passage obliquely or transversely to the shaft axis. This allows a particularly gentle deflection of the respective cord, so that forces arising when pulling on the cord on account of a deflection of the force from the distal to the circumferential direction are better supported by the cord. This minimizes the risk of the respective cord tearing. Preferably, a proximal portion of the first and/or second guide portions extends parallel to each other and to the shaft axis. The proximal portions may also be connected to each other and formed by a common groove-, channel- or slot-shaped reception. In a distal area of the guide portions (i.e. the portion of the first and/or second guide portion immediately proximal to the first or second passage, respectively), the guide portions preferably move away from each other in the circumferential direction. In other words, preferably the second guide portion, at least in the portion immediately proximal to the second through-hole, extends obliquely or transversely to the shaft axis with an inclination or circumferential direction opposite to that of the first guide portion. A transition between the region running parallel to the shaft axis and the region of the first and/or second guide portion(s) running obliquely or transversely to the shaft axis can be curved or kinked.

Preferably, the first and/or second guide portion form(s) a proximal passage at its respective proximal end, in which the first or second cord is guided radially inwards out of the first or second guide portion.

Preferably, the rotating sleeve or, possibly, the base sleeve forms a drive groove or annular groove, which is adapted to receive the first and second cords, at a specific axial position that corresponds to an axial position of the first and second passages. Thus, the cords can be wound up at a specific position and do not run the risk of jamming between the two sleeves. In addition, it is particularly easy to provide space for attaching the first and/or second cord to the rotating sleeve. That is, the first and/or second cord is preferably fastened to the rotating sleeve within the drive groove or ring groove.

Preferably, the base sleeve is a radially outer sleeve and the rotating sleeve is a radially inner sleeve. This enables particularly easy assembly. In addition, a portion of the endoscope whose radially outer elements rotate and could possibly damage a patient's tissue is minimized. The rotating mechanism may further include a protective or covering sleeve or film as a radially outermost layer. This allows the first and/or second guide portion to be covered or closed radially outwardly to prevent contamination or introduction of foreign bodies into the rotating mechanism and to minimize the risk of injuring tissue.

Preferably, the endoscope has electrical, hydraulic and/or empty lines as well as a working channel (i.e., tubes and cords) that extend through the shaft and the rotating mechanism to or through the endoscope head. In particular, the rotating mechanism provides a holding or positioning ring at its proximal region, in particular adjacent to the shaft, which determines the position of the lines with respect to the rotating sleeve. In this way, the tubes and cords can be prevented from crossing over when the rotating mechanism is turned. In other words, the tubes and cords are laid in an orderly fashion.

Preferably, the shaft forms a cavity at its distal end, which opens in a distal direction and is dimensioned such that there is space for rotation and/or circumferential motion of the lines and the working channel, which run through the shaft to the endoscope head and rotate together with the rotating sleeve in a distal area, or that the lines can be moved between different positions depending on the rotational position of the rotating sleeve. This ensures a particularly low-friction and low-interference operation.

Preferably, the shaft has, at least at its distal end, a groove- or slot-shaped cord reception that opens in the distal direction and in which the first and/or second cord is guided. That is to say, the first and/or second cord is/are introduced into the shaft in a (radial) sheath area of the shaft. In this way, the first and/or second cord, which do not rotate together with the rotating sleeve, do not get in the way of the rotating/moving lines, and a particularly low-friction, low-failure-rate operation can be ensured. The first and/or second cord can be guided in a sheath inside the shaft. This helps to avoid differences in length of the cords, which could result from the shaft bending. Furthermore, the lines in a distal shaft portion are preferably movable in the circumferential direction and are coated with a sliding layer or a sliding sheath. This makes it possible to minimize friction that arises from the rotation and motion of the lines and the working channel relative to the distal shaft area. Thus, a particularly low-friction, low-failure operation can be ensured.

Further preferably, a deflector for steering a distal endoscope portion is provided distally from the rotating mechanism and proximally from the endoscope head. A deflector is a shaft portion that is bendable at least to one side with respect to the shaft axis, and can be actuated by means of a control wire. In particular, the deflector has a plurality of segments that are connected in a bendable manner to one another and are preferably wedge-shaped. Furthermore, the endoscope preferably has a folding mechanism, which is arranged between the rotating mechanism, preferably distally from the deflector, and the endoscope head or is formed by the endoscope head and allows the endoscope head to be folded down with respect to the shaft axis. The folding mechanism is preferably operable by a separate control wire, in particular independently of the deflector. A maximum fold-down angle of the folding mechanism is preferably smaller than a maximum angle of curvature of the deflector.

Preferably, the first and/or second cord is provided as a drive cord exclusively for driving the rotating sleeve. Preferably, the first and/or second cord is/are provided separately from control wires for driving steering of the endoscope head, in particular separately to driving the deflector and/or the folding mechanism. Preferably, the first and second cords are guided and operable on a cord pulley provided in the proximal operating portion or endoscope handle. Preferably, the first and second cords are connected to each other at their proximal ends.

Preferably, all parts of the rotating mechanism are mounted to one another in such a way that they form a coherent assembly independently of the shaft and/or the endoscope head. Thus, the rotating mechanism can be pre-assembled and can be easily and inexpensively fixed to the shaft and to the distal endoscope portion as a finished assembly.

### DESCRIPTION OF FIGURES

The present disclosure is described below with reference to preferred embodiments. However, these are only illustrative in nature and are not intended to limit the scope of the present disclosure. Furthermore, equal reference signs are used for equal components in the description of the various embodiments.

FIG. 1 shows a perspective view of an endoscope according to the present disclosure.

FIG. 2 shows a distal endoscope portion of the endoscope according to FIG. 1 as well as a top view of an endoscope head of the endoscope.

FIGS. 3, 4 and 5 schematically show a rotating mechanism of the endoscope according to FIGS. 1 and 2 in different assembly states.

FIG. 6 shows a rotating sleeve of the rotating mechanism according to FIG. 3.

FIG. 7 shows a base sleeve of the rotating mechanism according to a second embodiment.

FIG. 8 shows a schematic arrangement of a shaft and a base sleeve.

FIG. 9 is a perspective view of the individual components of the rotating mechanism according to a third embodiment.

FIG. 10 shows the rotating mechanism according to FIG. 8 in an assembled state.

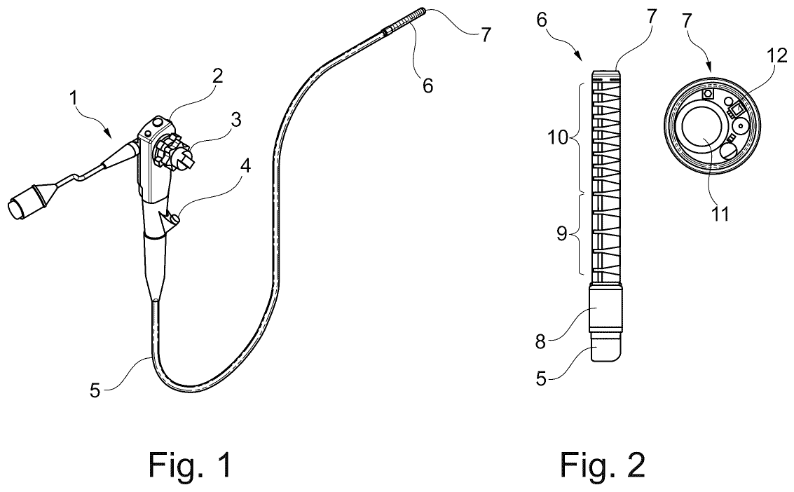

FIG. 1 shows a perspective view of an endoscope 1 according to the present disclosure. The endoscope 1 shown has a proximal endoscope operating portion or endoscope handle 2 with operating elements or wheels 3 and a Luer connector 4. A flexible shaft 5 extends in the distal direction from the endoscope operating portion or endoscope handle 2. A distal endoscope portion 6 is steerable to swivel and rotate an endoscope head 7, which forms a distal end of the endoscope 1.

FIG. 2 shows the distal endoscope portion 6 of the endoscope according to FIG. 1. A rotating mechanism 8 according to the disclosure is mounted on a distal end of the shaft 5, which will be described in more detail below with reference to the remaining figures. A distal end of the rotating mechanism 8 is connected to a deflector 9, which, as shown here by way of example, has several wedge-shaped segments that are hinged to one another on their broad sides and can be folded together on their narrow sides to curve the deflector 9. A folding mechanism 10 is mounted on the distal end of the deflector 9, which, as shown here by way of example, has several wedge-shaped segments that have a larger wedge angle than the segments of the deflector 9, in order to achieve a narrower angle of curvature than the deflector 9. The segments of the folding mechanism 10 are connected to each other and to the deflector 9 via the connecting plate. The deflector 9 and the folding mechanism 10 may also be formed differently. The endoscope head 7 is mounted at the distal end of the folding mechanism 10. Alternatively, the folding mechanism 10 can be considered as part of the endoscope head 7. Furthermore, FIG. 2 shows a top view of the endoscope head 7 of the endoscope 1. This forms an outlet of a working channel 11 and other elements, such as a camera or optics 12.

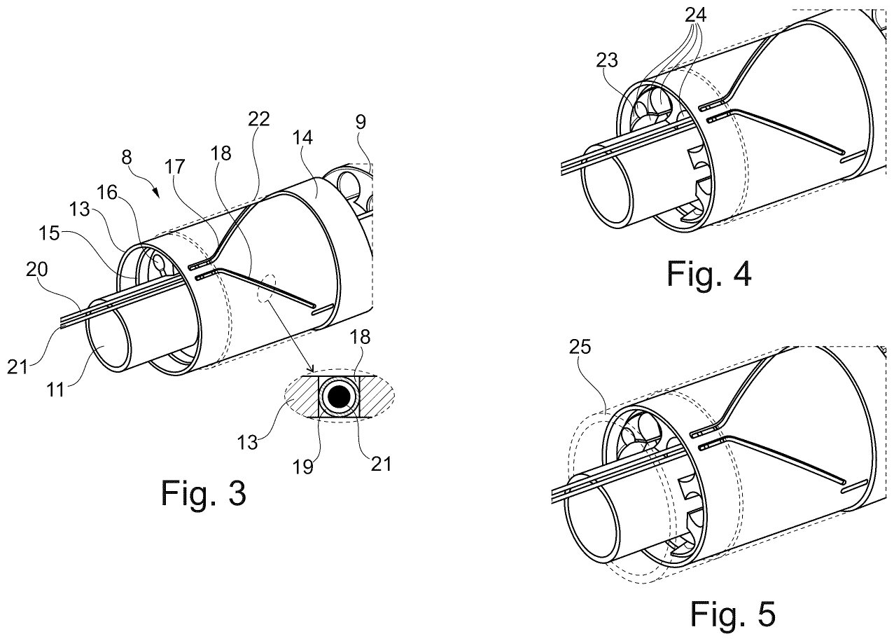

FIGS. 3, 4 and 5 show the rotating mechanism 8 according to FIGS. 1 and 2 in different assembly states, where, for reasons of clarity, in FIGS. 4 and 5, only reference signs of additional elements not shown in the previous figures (i.e. FIG. 3 or FIGS. 3 and 4) are incorporated, respectively. The remaining elements correspond to the elements shown in the previous figures. The rotating mechanism 8 has a base sleeve 13 that is adapted to be mounted on the distal end of the shaft 5. A rotating sleeve 14 is inserted into the base sleeve 13 and is rotatably mounted with respect to it, the distal end of the rotating sleeve 14 projecting beyond a distal end of the base sleeve 13 and being designed to be mounted on the deflector 9.

A holding or positioning ring 15 is arranged in an axially secure manner, in particular affixed, e.g. glued, on a proximal portion of the inside of base sleeve 13. The holding or positioning ring 15 is arranged proximally of the rotating sleeve 14 or of a step of the rotating sleeve 14 in order to form a stop for the rotating sleeve 14 in the proximal direction. That is to say, the rotating sleeve 14 can be inserted into a distal end of the base sleeve 13, but at most until a proximal end or the step of the rotating sleeve 14 hits the holding or positioning ring 15.

The rotating sleeve forms axial channels or openings 16, through which the working channel 11 (in FIGS. 3, 4 and 5 a part of it is shown as a tubular intermediate piece) as well as possibly further lines 32 (not shown in FIGS. 3, 4 and 5), are held in a fixed position in the rotating sleeve 14 and are guided in the axial direction through the rotating sleeve 14.

The base sleeve 13 forms a first guide portion 17 in the form of a groove- or slot-shaped reception, the proximal end portion 17a of which extends preferably parallel to a longitudinal shaft axis of the shaft 5. Further distally, the first guide portion 17 and the groove-or slot-shaped reception, respectively, bends and then extends obliquely in its/their middle portion and distal end portion 17b, i.e. extends distally along and in a first circumferential direction around the base sleeve 13. Furthermore, the base sleeve 13 forms a second guide portion 18 in the form of a groove- or slot-shaped reception, the proximal end portion 18a of which extends preferably parallel to the longitudinal axis of the shaft 5. Further distally, the second guide portion 18 and the groove- or slot-shaped reception, respectively, bends and its/their middle portion and distal end portion 18b then extend(s) obliquely in the distal direction and in a second circumferential direction opposite to the first circumferential direction such that it moves away from the first guide portion 17, i.e., the first guide portion 17 and the second guide portion 18 essentially are arranged in a Y-shape to one another, the proximal end portions 17a, 18a of the first and second guide portions 17, 18 forming the foot of the Y-shape and the middle portions and distal end portions 17b, 18b of the first and second guide portions 17, 18 forming the arms of the Y-shape.

A capillary tube 19 is attached in each of the first and second guide portions 17, 18, as is illustrated schematically in FIG. 3 in a detailed view of the second guide portion 18. A proximal or distal passage is formed, respectively, at a distal end and/or a proximal end of the first and/or second guide portion 17, 18, which deflects or opens the respective guide portion 17, 18 radially inwards in order to guide the first and second cords 20, 21 guided therein radially inwards, as described in more detail below. The respective proximal and/or distal passages can be formed by the corresponding distal and/or proximal end of the first and/or second guide portion 17, 18 being free of the capillary tubes 19, so that the respective groove- or slot-shaped reception of the first and/or second guide portions 17, 18 is connected at least to the inner circumferential surface of the base sleeve 13. Alternatively, the respective proximal and/or distal passages can be formed by portions of the capillary tubes 19 at the distal and/or proximal ends of the first and/or second guide portions being bent radially inwards.

A first cord 20 is guided through the shaft 5 and, starting from an inner side of the base sleeve 13, is introduced through the proximal passage provided there into the capillary tube 19 held in the first guide portion 17. The first cord 20 is deflected in the circumferential direction by the first guide portion 17 and exits at the distal end of the first guide portion 17 through the distal passage provided there to the inside of the base sleeve 13, in order to reach rotating sleeve 14 there, as is described in more detail below. A second cord 21 is guided in a manner corresponding to the first cord 20 through the second guide portion 18 in the distal direction and to rotating sleeve 14. Distal end portions of the first and second cords 20, 21 are attached to the rotating sleeve 14 and can be wound onto and unwound from it, as is described in more detail below.

A cover film or cover sleeve 22 is provided radially outside, which sits radially outside on the base sleeve 13 and at least partially covers the first and second guide portions from radially outside.

In FIG. 4, a locking plate or end disk 23 is additionally mounted on the arrangement from FIG. 3, which is inserted into the base sleeve 13 proximal to the holding or positioning ring 15 and is connected in a torque-proof manner to the rotating sleeve 14, e.g. screwed to it. The holding or positioning ring 15 forms a stop for the locking plate or end disk 23 in the distal direction. This means that when the rotating sleeve 14 is connected to the locking plate or end disk 23, axial motion of the rotating sleeve 14 relative to the base sleeve 13 is essentially prevented by the holding or positioning ring 15. Thus, the rotating sleeve 14 is rotatably mounted in the base sleeve 13 together with the locking plate or end disk 23 and is fixed in position in the axial direction by the holding or positioning ring 15. The rotating mechanism 8 thus forms an assembly that can be preassembled. The proximal passages of the first and second guide portions 17, 18 are arranged proximally of the locking plate or end disk 23 or are partially defined by it. The locking plate or end disk 23 also forms feed-through openings 24 for the lines 32 and the working channel 11 to fix their position in the rotating sleeve 14.

In FIG. 5, in addition to the arrangement from FIG. 4, a connecting ring or transition body 25 is provided, which is arranged proximally of the base body 13 in order to connect the latter to the shaft 5, e.g. by clamping, screwing, gluing or the like. In the present example, the connecting ring or transition body 25 covers the proximal passages of the first and second guide portions 17, 18 and, at its distal end, is directly in contact with the cover film or cover sleeve 22.

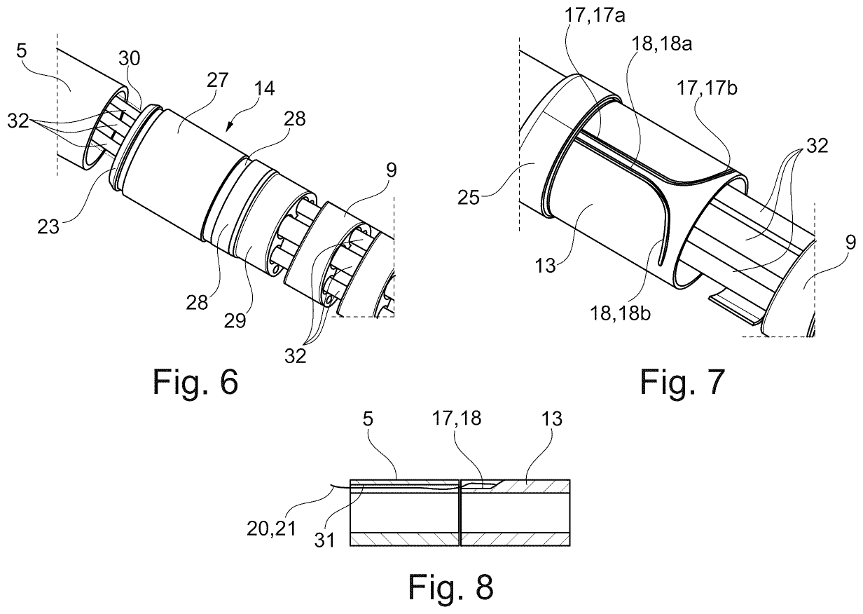

FIG. 6 shows the rotating sleeve 14 of the rotating mechanism 8 in its position between the shaft 5 and the deflector 9, as well as lines 32 guided through these three components. The rotating sleeve 14 can be the rotating sleeve according to FIG. 3, but it can also be part of a second embodiment. The locking plate or end disk 23 is attached proximally to the rotating sleeve 14. At its proximal end, the rotating sleeve 14 has a positioning portion 26 with a diameter that is reduced with regard to a distally adjacent bearing portion 27 and the locking plate or end disk 23, in order to provide a reception and stops for the holding or positioning ring 15. The rotating sleeve 14 is rotatably mounted on the base sleeve 13 via bearing portion 27. A circumferential, annular drive groove or annular groove 28 is formed distally from the bearing portion. It is adapted to receive and fasten the first and second cords 20, 21, which can thus be wound around/off the rotating sleeve 14 in the drive groove or annular groove 28. The cords 20, 21 can be glued into the drive groove or annular groove 28, for example in radial holes schematically shown here as dots.

A distal end of the rotating sleeve 14 forms a fastening portion 29, which is configured for fastening the deflector 9. A collar 30 is formed between the drive groove or annular groove 28 and the fastening portion 29, which collar defines an assembly position for the deflector 9 on its distal side and an assembly position for the cover film or cover sleeve 22 on its proximal side. Proximal to the rotating sleeve 14, i.e. within a distal portion of shaft 5 and optionally within a proximal portion of base sleeve 13, lines 32 are wrapped in a sliding foil 30 in order to improve their relative movement, i.e. a rotation and movement in the circumferential direction due to the rotation of rotating sleeve 14, with respect to stationary components of or in shaft 5.

FIG. 7 shows a base sleeve 14 and a connecting ring or transition body 25 of rotating mechanism 8 according to the second embodiment. This base sleeve 14 differs from the base sleeve 14 of the first embodiment essentially in the shape of the first and second groove- or channel-shaped guide portions 17, 18, which are groove- or slot-shaped receptions. As shown in FIG. 7, these run essentially parallel to one another and to the shaft axis in a proximal and middle region of guide portions 17, 18 and only curve in the circumferential direction at their distal end region. At their distal ends, guide portions 17, 18 and the groove- or slot-shaped receptions, respectively, extend essentially exactly in the circumferential direction, so that the guide portions 17, 18 essentially describe a 90° curve.

FIG. 8 schematically shows an arrangement of the shaft 5 relative to base sleeve 13 in a longitudinal section running through a proximal end of the first or second guide portion 17, 18. The shaft 5 forms, at least in its distal end region, a channel-, groove-, or slot-shaped cord reception 31, in which the first and second cords 20, 21 are received. Thus, the cords 20, 21 do not interfere with the movement of lines 32 and the working channel 11 within the shaft 5. Furthermore, the cords can be guided within the shaft 5 in a common sleeve or two separate sleeves.

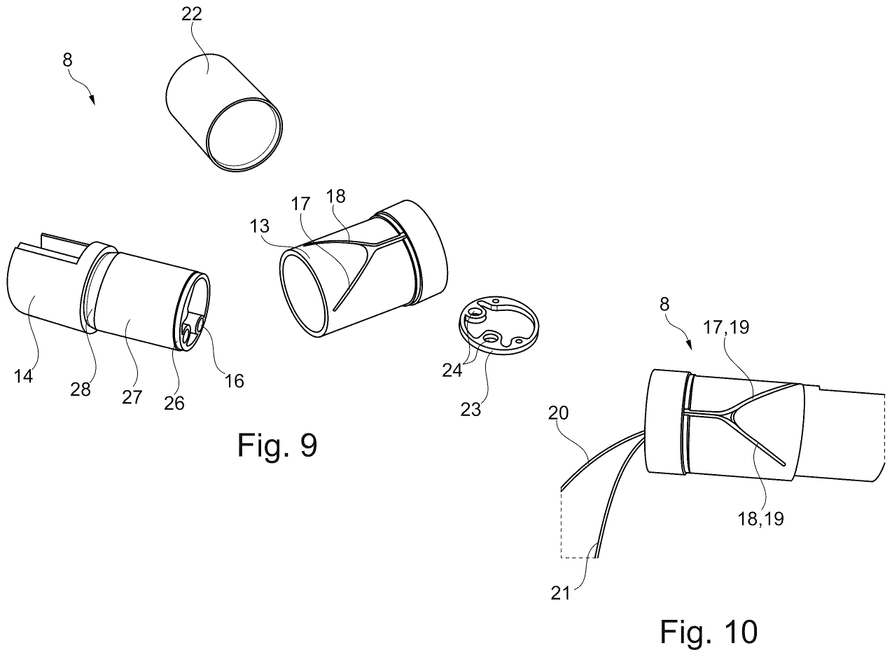

FIG. 9 shows a perspective view of the individual components of the rotating mechanism according to a third embodiment. One difference between this embodiment and the first embodiment is that no separate connecting ring or transition body 25 is provided, but that it is formed integrally with the base sleeve 13. Furthermore, the first and second guide portions 17, 18 and the groove- or slot-shaped receptions, respectively, are formed at their proximal ends, parallel to the shaft axis, as a single, common groove- or slot-shaped reception, which only divides further distally into the first and second guide portions 17, 18 in a Y-shape. Moreover, guide portions 17, 18 and the groove- or slot-shaped receptions are closed radially inside, except for the proximal and distal passages.

FIG. 10 shows the rotating mechanism according to FIG. 8 in an assembled state. The first and second cords 20, 21 are introduced in guide portions 17, 18 or in the capillary tubes held or attached therein.

### LIST OF REFERENCE SIGNS

1 Endoscope
2 Endoscope operating portion or endoscope handle
3 Operating elements or wheels
4 Luer connection
5 Flexible shaft
6 Distal endoscope portion
7 Endoscope head
8 Rotating mechanism
9 Deflector
10 Folding mechanism
11 Working channel
12 Camera or optics
13 Base sleeve
14 Rotating sleeve
15 Holding or positioning ring
16 Lines/openings for lines
17 First guide portion
17a Proximal end portion of the first guide portion
17b Distal end portion of the first guide portion
18 Second guide portion
18a Proximal end portion of the first guide portion
18b Distal end portion of the first guide portion
19 Capillary tube
20 First cord
21 Second cord
22 Cover film or cover sleeve
23 Locking plate or end disk
24 Feed-through openings
25 Connecting ring or transition body
26 Positioning portion
27 Bearing portion
28 Drive groove or annular groove
29 Fastening portion
30 Sliding foil/sliding sleeve
31 Cord reception
32 Lines

1-13. (canceled)

14. An endoscope comprising:
a shaft being essentially tubular and designed for insertion into a patient's body, said shaft having a shaft axis,
an endoscope head comprising a camera and an outlet of a working channel, the endoscope head being arranged distally from the shaft and being bendable or foldable with respect to the shaft axis,
a rotating mechanism arranged between the endoscope head and the shaft, the rotating mechanism being configured to rotate the endoscope head about the shaft axis with respect to the shaft, the rotating mechanism comprising:
(i) a base sleeve being connected to the shaft in a torque-proof manner with respect to the shaft axis, the base sleeve forming a first guide portion, the first guide portion being in the form of a groove-shaped or slot-shaped reception in a side wall of the base sleeve or on a surface of the side wall radially opposite a rotating sleeve, the base sleeve forming a first distal passage at a distal end of the first guide portion,
(ii) the rotating sleeve being connected to the endoscope head in a torque-proof manner with respect to the shaft axis, the rotating sleeve and the base sleeve being nested within one another, and
(iii) a first cord comprising a distal cord portion, the distal cord portion being attached to the rotating sleeve such that the first cord can be wound or unwound around the rotating sleeve, the first cord being guided by the first guide portion, the first cord being guided through the first distal passage from the first guide portion to the rotating sleeve.

15. The endoscope according to claim 14, wherein the endoscope comprises a capillary tube, the capillary tube being mounted in or on the first guide portion of the base sleeve in a manner such that the capillary tube is substantially fixed in position, the first cord being guided in the capillary tube.

16. The endoscope according to claim 14, wherein at least a portion of the first guide portion extends obliquely or transversely to the shaft axis directly proximal to the first distal passage.

17. The endoscope according to claim 14, wherein the endoscope comprises a second cord, and the base sleeve comprises a second guide portion, the second guide portion being in the form of a groove-shaped or slot-shaped reception,
the second cord being guided through the second guide portion in a distally extending direction and being wound or being able to be wound around the rotating sleeve in a circumferential direction opposite to the first cord.

18. The endoscope according to claim 17, wherein the base sleeve forms a second distal passage at the distal end of the second guide portion, the second distal passage being spaced apart from the first distal passage in the circumferential direction, the second cord being guided through the second distal passage.

19. The endoscope according to claim 18, wherein the second guide portion extends obliquely or transversely to the shaft axis at least in a portion directly proximal to the second distal passage and with either an inclination or direction opposite to that of the first guide portion.

20. The endoscope according to claim 18, wherein the rotating sleeve or the base sleeve forms a drive groove or annular groove at a specific axial position, the specific axial position corresponding to an axial position of the first and second distal passages.

21. The endoscope according to claim 14, wherein the base sleeve is a radially outer sleeve and the rotating sleeve is a radially inner sleeve.

22. The endoscope according to claim 14, wherein the shaft has, at least at a distal end of the shaft, a groove-shaped, channel-shaped or slot-shaped cord reception, in which at least the first cord is guided.

23. The endoscope according to claim 14, further comprising electrical, hydraulic and/or empty lines, the electrical, hydraulic and/or empty lines extending through the shaft and the rotating mechanism to or through the endoscope head, wherein a distal end of the shaft forms a cavity dimensioned such that the electrical, hydraulic and/or empty lines are movable between different positions that depend on a rotational position of the rotating sleeve.

24. The endoscope according to claim 23, wherein the rotating mechanism comprises either a holding ring or a positioning ring adjacent to the shaft, the holding or positioning ring determining a position of the electrical, hydraulic and/or empty lines with respect to the rotating sleeve.

25. The endoscope according to claim 23, wherein the electrical, hydraulic and/or empty lines are movable in a distal shaft portion in a circumferential direction and are coated by a sliding layer or a sliding sleeve.

26. The endoscope according to claim 14, wherein the endoscope comprises a deflector for deflecting a distal endoscope portion, the deflector being arranged distally of the rotating mechanism and proximally of the endoscope head,
the endoscope further comprising a folding mechanism, the folding mechanism being arranged between the deflector and the endoscope head, or being formed by the endoscope head, wherein the folding mechanism is operable independently of the deflector in order to fold the endoscope head down to one side with respect to the shaft axis.
